(19) United States
(12) Patent Application Publication
(10) Pub. No.: US 2005/0271597 A1
(43) Pub. Date: Dec. 8, 2005

(54) Prostate hypertrophy treatment composition and method

(76) Inventor: Alec D. Keith, Hilo, HI (US)

Correspondence Address: THE WEBB LAW FIRM, P.C., 700 KOPPERS BUILDING, 436 SEVENTH AVENUE, PITTSBURGH, PA, 15219, US

(21) Appl. No.: 11/057,067

(22) Filed: Feb. 11, 2005

Related U.S. Application Data
(60) Provisional application No. 60/544,387, filed on Feb. 13, 2004.

(51) Int. Cl. A61L 9/04; A61K 9/14; A61K 31/57

(52) U.S. Cl. 424/46; 514/177

(57) ABSTRACT

The present invention provides a method and composition for treatment of benign prostate hyperplasia (BPH) in men via a transscrotal delivery system. The composition of the present invention includes the steroid hormone progesterone containing permeation enhancers that greatly facilitate permeation through the skin, thus preventing modification of the constituents therein and providing continuous and sustained delivery of progesterone for several hours that mimics the circadian rhythm of endogenous progesterone. The progesterone composition preferably is capable of delivering an effective dosage amount of about 65-100 mg of progesterone per ml when applied directly onto the surface of scrotum.

The present application claims priority to U.S. Provisional Application No. 60/544,387, filed Feb. 13, 2004, which is incorporated herein by reference.

### BACKGROUND OF THE INVENTION

1. Field of the Invention

The invention relates to methods and compositions for transscrotal delivery at therapeutically effective rates of biologically active agents having improved permeation rates and efficacy for the treatment of prostate disease.

2. Description of Related Art

Benign prostate hyperplasia (BPH) is an enlargement of the prostate beginning from the so-called “inner” prostate. Statistics show that 50% of men over the age of 40 will experience problems with their prostate. One in 11 American men over age 50 will develop prostate cancer. By the age of 70, over 60% of American men will have BPH, and by age 80, the number goes up to 85%. The attendant symptoms are due primarily to the resultant obstructions of the urethra. Voiding is made difficult and retention of urine residue occurs. Retained urine at first can interfere with sleep as the sufferer wakes up during the night. At other times, pressure may make it impossible to control urine flow properly and the sufferer experiences incontinence. Retained urine in the bladder can allow bacterial growth and infection and the urine may flow back up the ureters and cause kidney infection. Without surgical intervention, uremia can ensue.

BPH can be considered an aspect of male menopause because an increased ratio of the sex steroids estrogen to testosterone is active in BPH just as, conversely, women passing through menopause experience an increased ratio of testosterone to estrogen.

Testosterone levels in the male are at a peak at adolescence and decrease thereafter, with the rate of decrease increasing sharply by about age 50. Such decreases in circulating levels of testosterone produce a compensatory release of stimulatory hormones for testosterone production, which is ineffective to increase testosterone levels. The stimulatory hormones, however, cause an elevated rate of transformation of testosterone into 5-alpha-dihydrotestosterone (DHT) via upregulation of 5-alpha reductase, and/or to an increased binding and/or decreased clearance of DHT from prostate cells. DHT binds to androgen receptors on prostate cells and then is transported into the nucleus of the cells where it affects DNA expression, resulting in prostatic growth. Current research indicates that DHT is a necessary, but not the sole cause in the etiology of BPH. The steroid hormone, progesterone, is the chief inhibitor of 5-alpha reductase, and during the aging process progesterone levels fall in men, especially after age 60. This decrease in progesterone levels results in an increased conversion of testosterone to DHT, which can result in BPH. The administration of progesterone to treat BPH is well known, and several routes of administration have been disclosed. Oral or intravenous administration of steroid hormones such as progesterone is not practical and generally not available in the United States. Furthermore, there are a number of medications which in at least some patients are not tolerated well when orally administered, in that they may cause undesirable gastrointestinal or other side effects and/or which may provide undesirably high or low concentrations or delayed concentrations in a target tissue. In some cases, dosages that are appropriate for oral administration, upon being distributed more or less uniformly throughout the body, are undesirably low in a particular tissue to achieve desired results. Oral or injection administration may result in a too slow or too rapid increase in blood plasma levels, e.g., may involve an undesirably long time delay as the active agent is absorbed by the digestive system before entering the bloodstream, or may result in a “spike” in blood plasma levels followed by an undesirably low level, whereas a more constant level would be preferable.

The most common route of administration of steroid hormones is intramuscular depot injections. This route has the advantage of ensuring delivery to the patient without concerns of patches slipping off or doses being forgotten. A significant problem with intramuscular injections of hormones, however, is the rapid serum peak to supraphysiologic levels, reached between hours to a few days after injection, then a gradual decline over the following weeks. Thus, there is no circadian variation in delivery.

The transdermal route of parenteral delivery of active agents, such as progesterone, has been shown to offer distinct advantages over conventional administration methods, such as avoidance of the first-pass elimination by the liver, safe drug levels in the blood, easy termination of drug delivery, high efficacy and improved patient compliance. Indeed, topical products have been used for centuries for treating local skin disorders. Numerous devices are known to the art for delivering various drugs and other biologically active agents through the skin, as disclosed, for example, in representative U.S. Pat. Nos. 4,144,317, 4,704,282, 5,879,322, 6,444,234 and 6,572,880, incorporated herein by reference.

Transdermal delivery systems, however, have been accompanied by their own side effects, including a potential for skin irritation that can arise from the gel or other matrix, or from the active agent itself, or from the interaction of the active agent with the matrix. Additionally, a transdermal system must be configured such that the combination of the matrix and the active agent does not react with or modify the active agent, or otherwise render it ineffective. Transdermal systems also need to provide sufficient diffusion coefficients, such that the delivery system is not adversely affected by expected temperature variations during normal manufacture, transportation, storage and use, such that the gel or other matrix retain the desired viscosity, and such that the active agent can be properly dispersed or dissolved in the matrix so that the components remain homogeneous. Furthermore, because the skin is the largest metabolizing organ in the body with respect to surface area, certain agents that are delivered transdermally may be metabolized into inactive or potentially harmful metabolites, and/or other agents may not be capable of permeating the skin because of low permeability properties of the agent.

Prior art transdermal delivery systems primarily have consisted of an agent reservoir which may be merely a mixture of a matrix material containing dissolved or dispersed agent which is maintained on the skin for a predetermined period of time by an adhesive or other means, such as a patch, pad or strap. These systems, however, often lose their adhesive properties and loosen or fall off before the predetermined administration period of the active agent is completed. To deal with this problem, some products are sold with adhesive overlays that are applied on top of the device and extend beyond its original borders to keep the device in place for the remainder of the administration period.

Other products use adhesives that are overly aggressive, i.e., as close to being too painful to remove as possible without actually being too painful for consumer tolerance. Still other transdermal delivery devices, such as those disclosed in U.S. Pat. Nos. 4,704,282, 4,725,439 and 4,867,982, have been designed to be applied to sensitive skin sites, such as the scrotum or breast. These devices have skin-contacting surfaces that have a low level of tack in order to allow them to cling in an almost non-adhesive manner to the skin. Testoderm™ transdermal testosterone represents this type of product and users are instructed that it can be removed while showering or swimming, for example, and then replaced. An improved version of Testoderm™ with adhesive has thin strips of polyisobutylene adhesive applied to about 12% of the surface of the device to assist in maintaining the device on the scrotum.

Transdermal delivery of particular active agents, such as hormones, via scrotal tissue is advantageous over non-scrotal tissue because of the higher permeation rate of the scrotum. The sensitivity of scrotal tissue, however, imposes significant constraints on the characteristics that a transdermal system for scrotal administration of active agents must have. For example, the system must be sufficiently thin and flexible so as to be able to conform easily to the configuration of the scrotal sac and must be sufficiently adherent to be able to maintain itself in place without being so adherent as to create discomfort on removal.

Thus, there exists a need for a transdermal delivery system for the prevention or treatment of pathological conditions, such as BPH, which is capable of delivering effective dosage amounts of an active agent, such as progesterone, into the systemic circulation in a sustained manner that closely mimics natural circadian levels of the agent without altering its potency and without the need for painful and cumbersome devices.

### SUMMARY OF THE INVENTION

The present invention provides a method and composition for treatment of benign prostate hyperplasia (BPH) in men via a transscrotal delivery system. The composition includes a progesterone steroid hormone that exhibits a surprisingly high permeation rate through the scrotal sac along with a sustained delivery that closely mimics circadian levels of progesterone in vivo. Additionally, the high permeation rate prevents undesirable modifications of the hormone within the skin.

The present invention also provides a method for treating BPH by applying the progesterone composition directly onto at least a portion of the outer surface of the scrotum. The progesterone composition is formulated to deliver an effective dosage amount of about 65-100 mg of progesterone per dose, with about 1-10 ml of the composition being applied about every twelve hours. The concentration of progesterone in the progesterone composition therefore can range from between about 0.65-10 mg/ml up to between about 65-100 mg/ml.

The progesterone composition is absorbed rapidly across the scrotal sac where the progesterone hormone is stored in adipose tissue. After the progesterone reaches a saturation level in the adipose cells, it then diffuses into the circulatory system for eventual uptake by the prostate gland. One application of the progesterone composition about every twelve hours is believed, without being bound by the theory, to maintain a steady-state concentration of progesterone in the system that closely mimics the normal circadian rhythm of endogenous progesterone.

The transscrotal delivery of the progesterone composition of the present invention can be formulated either into a cream or into a liquid spray for use in the treatment of BPH.

### DETAILED DESCRIPTION OF THE INVENTION

The present invention provides a method and composition for treatment of benign prostate hyperplasia (BPH) in men via a transscrotal delivery system. The composition includes a progesterone steroid hormone that exhibits an exceptionally high permeation rate through the scrotal sac along with a sustained delivery that closely mimics the circadian levels of progesterone in vivo. Additionally, the high permeation rate prevents undesirable modification of the hormone.

The present invention also provides a method for treating BPH by directly applying the progesterone composition onto at least a portion of the surface of the scrotum twice daily, preferably at twelve hour intervals. The transscrotal delivery of the progesterone composition of the present invention can be formulated into a cream or liquid spray for use in the treatment of BPH.

In one embodiment of the present invention, the progesterone composition can include, without limitation, about 0.1-10 vol. %, preferably about 0.5-5.0 vol. %, and most preferably about 1 vol. % of micronized progesterone, combined with effective carrier vehicles, such as about 1-8 vol. %, preferably about 3-6 vol. % and most preferably about 5 vol. % of mineral oil or squalene oil; rheology modifiers, i.e., suspension agents and stabilizers, such as about 0.1-1 vol. %, preferably about 0.2-0.8 vol. %, and most preferably about 0.5 vol. % of Carbopol 934; emulsifiers, such as about 1-5 vol. %, preferably about 2-4 vol. %, and most preferably about 3 vol. % polyethylene glycol 100 stearate; about 1-5 vol. %, most preferably about 2-4 vol. %, and most preferably about 3 vol. % of glycerol stearate; about 1-5 vol. %, preferably about 2-4 vol. %, and most preferably about 3 vol. % PEG 40 stearate; about 0.5-5 vol. %, preferably about 1-4 vol. %, and most preferably about 3 vol. % of lanoline alcohol and about 0.1-1 vol. %, preferably about 0.2-0.8 vol. %, and most preferably about 0.5 vol. % of dialkylsodium sulfonate; humectants, such as about 1-5 vol. %, preferably about 2-4 vol. %, and most preferably about 3 vol. % of propylene glycol, and up to about 5 vol. % triethylolamine; emollients, such as about 0.5-4 vol. %, preferably about 1-3.5 vol. %, and most preferably about 3 vol. % of lanolin; preservatives, such as about 0.1-1 vol. %, preferably about 0.2-0.9 vol. %, and most preferably about 0.8 vol. % of imidazolidinyl urea, about 0.01-2 vol. %, preferably about 0.05-1.8 vol. %, and most preferably about 1.5 vol. % of methylparaben or about 0.01-2 vol. %, preferably about 0.05-1.8 vol. %, and most preferably about 1.5 vol. % of ethylparaben; and metal sequestrants, such as about 0.01-5 vol. %, preferably about 0.05-4 vol. %, and most preferably about 3 vol. % of ethylenediaminetetraacetic acid.

The progesterone composition also can contain, without limitation, about 0.1-1 vol. %, preferably about 0.2-0.8 vol. %, and most preferably about 0.5 vol. % of ascorbic acid, ascorbial palmitate, BHT, lecithin, beta-carotene, colloidal silver, neem oil, dimethylsulfoxide, coconut oil, EMU oil or a combination thereof.

The main permeation barrier for most active agents is the stratum corneum, which is typically only 10-15 μm thick and consists of several layers of non-viable cornified cells. The interstices between these corneocytes are filled with several lipid bilayers. The intercellular lipid bilayers are the most important lipophilic pathway for drug absorption. This lipid barrier more or less dictates the physico-chemical characteristics of a drug to be a candidate for transdermal absorption.

The enhanced permeation rate of the progesterone composition through the scrotum is achieved by the inclusion of permeation rate enhancers, which include, without limitation, polyethylene glycols, peptide/fatty acid complexes with about 10-20 carbon rings, and preferably with about 12 to 18 carbon rings, or certain mono, di, or triglycerides of fatty acids. The permeation rate enhancers can be present in concentrations ranging from between about 0.01-20 vol. %, and preferably about 10 vol %.

The permeation rate enhancers allow for rapid absorption of the progesterone composition across the scrotal sac, after which the lipophilic progesterone hormone is stored in adipose cells. After the progesterone hormone reaches a saturation level in the adipose cells, it diffuses into the circulatory system for eventual uptake by the prostate gland. The present invention therefore can deliver a sustained effective dosage amount of progesterone to the prostate gland.

The progesterone cream or liquid is formulated to deliver an effective dosage amount of about 65-100 mg of progesterone per dose, with about 1-10 ml of the composition being applied about every twelve hours. The concentration of progesterone in the progesterone composition can therefore range from about 0.65-10 mg per ml to 65-100 mg per ml.

In one embodiment of the present invention, the concentration of progesterone in the composition is 65-100 mg per ml, with 1 ml of progesterone cream or liquid being applied to the entire surface of the scrotum every twelve hours, thus delivering 65-100 mg of progesterone in the twelve hour period. This effective dosage amount is continuously released into the systemic circulation, reaching peak levels after about two hours, with levels gradually decreasing to about half after twelve hours. One application of the progesterone compound every twelve hours is believed, without being bound by the theory, to maintain a steady-state concentration of progesterone in the system that closely mimics the normal circadian rhythm of endogenous progesterone.

It should be understood that the following example and embodiment described herein are for illustrative purposes only and that various modifications or changes in light thereof will be suggested to persons skilled in the art and are to be included within the spirit and purview of this application.

### EXAMPLE 1

### Preparation and Application of the Transscrotal Progesterone Composition

A transscrotal progesterone composition was prepared by admixing 1 vol. % of micronized progesterone with 5 vol. % of mineral oil, 0.5 vol. % of Carbopol 934; 3 vol. % polyethylene glycol 100 stearate; 3 vol. % of glycerol stearate; 3 vol. % PEG 40 stearate; 3 vol. % of lanoline alcohol; 0.5 vol. % of dialkylsodium sulfonate; 3 vol. % of propylene glycol, 5 vol. % triethylolamine; 3 vol. % of lanolin; 0.8 vol. % of imidazolidinyl urea, 1.5 vol. % of methylparaben and 3 vol. % of ethylenediaminetetraacetic acid. The above constituents were emulsified in water (55.7 vol. %) to create a lotion dosage form containing 1% progesterone. One male subject was treated by applying 1 ml of the lotion, containing 65 mg progesterone, to the entire outer surface of the scrotum. The blood level of progesterone in the subject remained at about 0.1 ng/ml for six hours post-administration. On a different day, three male subjects including the one male subject described above were treated by applying 1 ml of the lotion, each ml containing 65 milligrams, to the entire outer scrotal surface. One hour after administration, the blood levels of progesterone were 0.9, 1.2 and 1.3 ng/ml, respectively; two hours after administration the blood levels of progesterone were 3.0, 3.5 and 3.8 ng/ml, respectively, and six hours after administration the blood levels of progesterone were 2.5, 3.0 and 3.5 ng/ml.

1. A transscrotal delivery system method for treating benign prostate hyperplasia in men in need of said treatment, comprising:
admixing a progesterone hormone with:
(i) at least one pharmaceutically effective excipient to make a progesterone composition suitable for application to scrotal tissue;
(ii) at least one permeation rate enhancer; and
applying an effective dosage amount of the progesterone composition directly onto at least a portion of the outer surface of the scrotum approximately every twelve hours, wherein said application mimics the normal circadian rhythm of endogenous progesterone.

2. The method of claim 1, wherein the progesterone hormone is a micronized progesterone, said micronized progesterone admixed in an amount ranging from between about 0.1-10 vol. %.

3. The method of claim 1, wherein the progesterone hormone is a micronized progesterone, said micronized progesterone admixed in an amount ranging from between about 0.5-5 vol. %.

4. The method of claim 1, wherein the progesterone hormone is a micronized progesterone and wherein the micronized progesterone is admixed in an amount of about 1 vol. %.

5. The method of claim 1, wherein the at least one pharmaceutically acceptable excipient is selected from the group consisting of effective carrier vehicles selected from the group consisting of about 1-8 vol. % mineral oil and 1-8 vol. % squalene oil; rheology modifiers (suspension agents and stabilizers), such as about 0.1-1 vol. % Carbopol 934; emulsifiers selected from the group consisting of about 1-5 vol. % polyethylene glycol 100 stearate, about 1-5 vol. % glycerol stearate, about 1-5 vol. % PEG 40 stearate, about 0.5-5 vol. % lanoline alcohol and about 0.1-1 vol. % dialkylsodium sulfonate; humectants selected from the group consisting of about 1-5 vol. % propylene glycol and up to about 5 vol. % triethylolamine; emollients, such as about 0.5-4 vol. % lanolin; preservatives selected from the group consisting of about 0.1-1 vol. % imidazolidinyl urea, about 0.01-2 vol. % methylparaben and about 0.01-2 vol. % ethylparaben; and metal sequestrants, such as about 0.01-5 vol. % ethylenediaminetetraacetic acid.

6. The method of claim 1, wherein the at least one pharmaceutically acceptable excipient is selected from the group consisting of effective carrier vehicles selected from the group consisting of about 3-6 vol. % mineral oil and 3-6 vol % squalene oil; rheology modifiers (suspension agents and stabilizers), such as about 0.2-0.8 vol. % Carbopol 934; emulsifiers selected from the group consisting of about 2-4 vol. % polyethylene glycol 100 stearate, about 2-4 vol. % glycerol stearate, about 2-4 vol. % PEG 40 stearate, about 1-4 vol. % lanoline alcohol and about 0.2-0.8 vol. % dialkylsodium sulfonate; humectants selected from the group consisting of about 2-4 vol. % propylene glycol and up to about 5 vol. % triethylolamine; emollients, such as about 1-3.5 vol. % lanolin; preservatives selected from the group consisting of about 0.2-0.9 vol. % imidazolidinyl urea, about 0.05-1.8 vol. % methylparaben and about 0.05-1.8 vol. % ethylparaben; and metal sequestrants, such as about 0.05-4 vol. % ethylenediaminetetraacetic acid.

7. The method of claim 1, wherein the at least one pharmaceutically acceptable excipient is selected from the group consisting of effective carrier vehicles selected from the group consisting of about 5 vol. % mineral oil and 5 vol % squalene oil; rheology modifiers (suspension agents and stabilizers), such as about 0.5 vol. % Carbopol 934; emulsifiers selected from the group consisting of about 3 vol. % polyethylene glycol 100 stearate, about 3 vol. % glycerol stearate, about 3 vol. % PEG 40 stearate, about 3 vol. % lanoline alcohol and about 0.5 vol. % dialkylsodium sulfonate; humectants selected from the group consisting of about 3 vol. % propylene glycol and up to about 5 vol. % triethylolamine; emollients, such as about 3 vol. % lanolin; preservatives selected from the group consisting of about 0.8 vol. % of imidazolidinyl urea, about 1.5 vol. % methylparaben and about 1.5 vol. % of ethylparaben; and metal sequestrants, such as about 3 vol. % ethylenediaminetetraacetic acid.

8. The method of claim 5, wherein the progesterone composition also contains about 0.1-1 vol. % of the following agents selected from the group consisting of ascorbic acid, ascorbial palmitate, BHT, lecithin, beta-carotene, colloidal silver, neem oil, dimethylsulfoxide, coconut oil, EMU oil and any combination thereof.

9. The method of claim 6, wherein the progesterone composition also contains about 0.2-0.8 vol. % of the following agents selected from the group consisting of ascorbic acid, ascorbial palmitate, BHT, lecithin, beta-carotene, colloidal silver, neem oil, dimethylsulfoxide, coconut oil, EMU oil or any combination thereof.

10. The method of claim 7, wherein the progesterone composition also contains about 0.5 vol. % of the following agents selected from the group consisting of ascorbic acid, ascorbial palmitate, BHT, lecithin, beta-carotene, colloidal silver, neem oil, dimethylsulfoxide, coconut oil, EMU oil or any combination thereof.

11. The method of claim 1, wherein the at least one permeation enhancer is selected from the group consisting of polyethylene glycols, peptide/fatty acid complexes with about 10-20 carbon rings and mono, di, or triglycerides of fatty acids, and wherein the at least one permeation rate enhancer is present in concentrations ranging from between about 0.01-20 vol. %.

12. The method of claim 1, wherein the at least one permeation enhancer is selected from the group consisting of polyethylene glycols, peptide/fatty acid complexes with about 12-18 carbon rings, and preferably with about 12 to 18 carbon rings, and mono, di, or triglycerides of fatty acids, and wherein the at least one permeation rate enhancers is present in concentrations of 10 vol. %.

13. The method of claim 1, wherein the effective dosage amount of the progesterone composition is about 65-100 mg progesterone.

14. The method of claim 1, wherein the concentration of progesterone in the progesterone composition ranges from between about 6.5-10 mg/ml.

15. The method of claim 1, wherein the concentration of progesterone in the progesterone composition ranges from between about 65-100 mg/ml.

16. The method of claim 1, wherein about 1 to 10 ml of the progesterone composition is applied directly onto the outer surface of the scrotum about every twelve hours.

17. The method of claim 1, wherein the progesterone composition is formulated into a cream.

18. The method of claim 1, wherein the progesterone composition is formulated into a liquid spray.

19. The method of claim 18, wherein the liquid spray formulation of the progesterone composition contains a polymer binder selected from the group consisting of polyacrylate, polyester, and silicone.

20. A transscrotal delivery system method for treating benign prostate hyperplasia, comprising:
admixing about 1 vol % of a micronized progesterone hormone with:
(i) at least one pharmaceutically effective excipient selected from the group consisting of effective carrier vehicles selected from the group consisting of about 5 vol. % mineral oil and about 5 vol % squalene oil; rheology modifiers (suspension agents and stabilizers), such as about 0.5 vol. % Carbopol 934; emulsifiers selected from the group consisting of about 3 vol. % polyethylene glycol 100 stearate, about 3 vol. % glycerol stearate, about 3 vol. % PEG 40 stearate, about 3 vol. % lanoline alcohol and about 0.5 vol. % dialkylsodium sulfonate; humectants selected from the group consisting of about 3 vol. % propylene glycol and up to about 5 vol. % triethylolamine; emollients, such as about 3 vol. % lanolin; preservatives selected from the group consisting of about 0.8 vol. % of imidazolidinyl urea, about 1.5 vol. % methylparaben and about 1.5 vol. % of ethylparaben; and metal sequestrants, such as about 3 vol. % ethylenediaminetetraacetic acid, and about 0.5 vol. % of the following agents selected from the group consisting of ascorbic acid, ascorbial palmitate, BHT, lecithin, beta-carotene, colloidal silver, neem oil, dimethylsulfoxide, coconut oil, EMU oil and combination thereof, in order to make a progesterone composition suitable for application to scrotal tissue;
(ii) at least one permeation rate enhancer selected from the group consisting of about 10 vol. % of polyethylene glycols, peptide/fatty acid complexes with about 12-18 carbon rings and mono, di, or triglycerides of fatty acids; and
applying about 1 ml of the progesterone composition directly onto the outer surface of the scrotum, wherein the concentration of progesterone in the progesterone composition ranges from between about 65-100 mg/ml.

21. A progesterone composition made according to the method of claim 1.
